(19) United States
(12) Patent Application Publication
(10) Pub. No.: US 2025/0241527 A1
(43) Pub. Date: Jul. 31, 2025

(54) RIGID, CURVED LARYNGOSCOPE

(71) Applicant: UNIVERSITÄT ULM, Ulm (DE)

(72) Inventors: Patrick SCHULER, Ulm (DE); Thomas HOFFMANN, Ulm (DE); Leon SCHILD, Ulm (DE); Linus KIENLE, Ulm (DE); Felix BÖHM, Ulm (DE)

(73) Assignee: UNIVERSITÄT ULM, Ulm (DE)

(21) Appl. No.: 18/689,178

(22) PCT Filed: Sep. 1, 2022
(86) PCT No.: PCT/EP2022/074337

§ 371 Date: Mar. 5, 2024

(30) Foreign Application Priority Data
Sep. 9, 2021 (DE) 10 2021 209 981.5

(51) Int. Cl. A61B 1/267; A61B 1/00; A61B 1/018; A61B 1/05; A61B 1/06; A61B 17/02

(52) U.S. Cl. CPC A61B 1/267; A61B 1/00029; A61B 1/00066; A61B 1/00071; A61B 1/018; A61B 1/051; A61B 1/0676; A61B 1/0684; A61B 17/0218

(57) ABSTRACT

The invention relates to a rigid, curved laryngoscope. The laryngoscope is characterised in that it has a rigid, curved body, a light source and image-capturing unit integrated in the body and two channels, each suitable for accommodating a flexible surgical instrument, which is suited for a mechanical intervention at the operation site. The laryngoscope according to the invention can be simply and economically produced and is suited for repeated use. It is also mechanically stable and can be handled quickly and easily.

A rigid, curved laryngoscope is provided. The laryngoscope is characterized in that it has a rigid, curved body, a light source and image capturing unit integrated into the body, and two channels, each of which is suitable for receiving a flexible surgical instrument which is suitable for a mechanical intervention at the site of surgery. The laryngoscope according to the invention is easy and inexpensive to manufacture and is suitable for repeated use. It is also mechanically stable and can be handled quickly and easily. It also enables a wide range of different, flexible surgical instruments to be used, which can be moved to the site of surgical intervention in a targeted and simple manner.

Benign and malignant pathologies in the larynx and pharynx are common diseases. Transoral surgery is often indicated for diagnosis and treatment, for which microlaryngoscopic techniques such as transoral laser microsurgery (TLM) have become the gold standard. This procedure is usually performed using a rigid, straight surgical laryngoscope, which requires the patient's cervical spine to be placed in an hyperextended position. In the correct position, the surgical laryngoscope is fixed with a chest support. The surgical area is visualized by using an surgical microscope, while an external CO₂ laser beam attached to the microscope and straight microsurgical instruments enable the excision of pathological structures in the larynx and pharynx. This approach produces good results for most patients, an acceptable functional outcome and few complications.

Nevertheless, there are cases in which the use of TLM is unsatisfactory or even impossible due to insufficient laryngeal exposure. Due to the rigid and straight form of the standard surgical laryngoscope, the straight microsurgical instruments and the need for a straight line of sight for the laser beam, the cervical spine needs to be in a hyperextended position in order to gain direct access to the larynx. This is impractical, particularly for patients with immobilized cervical vertebrae, insufficient mouth opening, prominent upper jaw, pathologically enlarged tongue or scarring after undergoing radiotherapy.

Various types of hyperangulated video laryngoscopes with an integrated camera and light unit are available for visualizing the vocal cords. However, these video laryngoscopes only allow visualization of the pharyngeal area during intubation, as these laryngoscopes do not have working channels for surgical instruments. Apart from this, these laryngoscopes are not configured for surgical use, as they do not provide the option of intraoperative fixing. In fact, the physician needs to hold these devices by hand during use, which is not feasible in a surgical environment and prevents bimanual handling with surgical instruments.

US 2009/0264708 A1 discloses a device for inserting an anesthetic tube. In this device, an endoscope and a light unit have to be used separately to enable visualization of the surgical area, which is time-consuming and laborious. The device is also intended for single use which makes the device unecological and uneconomical. As it is intended for single use, this device is also not suitable for chemical sterilization. Furthermore, this device does not provide an option for fixing the device intraoperatively to a frame.

US 2014/0316207 A1 discloses a laryngoscope for microlaryngoscopy of the larynx with longitudinal working channels. However, this laryngoscope is flexible, which makes it more prone to damage and more difficult to handle. For example, this laryngoscope has to be fixed in the desired position by using inflatable cushions, otherwise it will not remain in the desired position. The inflation and deflation of the cushions is time-consuming and labor-intensive. The cushions are also prone to damage from the effects of mechanical force. For visualizing the surgical area it is necessary with this laryngoscope to insert a fiber optic through the working channels of the laryngoscope, which also makes it more difficult to handle.

U.S. Pat. No. 10,285,571 B2 discloses an S-shaped laryngoscope with working channels. With this laryngoscope it is necessary to guide a conventional endoscope and a light source through the device to enable a visualization of the surgical area. The working channels of this laryngoscope are configured to be straight so that they do not provide any medialization of the surgical instruments. The laryngoscope also cannot be fixed to a stable frame, which jeopardizes the secure positioning of the laryngoscope in the desired location and makes bimanual handling with surgical instruments more difficult. This laryngoscope is also not suitable for chemical sterilization.

Schild, L. R. et al. (“Adding Flexible instrumentation to a curved videolaryngoscope: A novel tool for laryngeal surgery”, Laryngoscope, 00:1-8, 2020) disclose a curved, rigid laryngoscope, which has a light source (LED light source) and a video chip for recording the surgical area. In addition, this laryngoscope has two working channels consisting of a PTFE tube for receiving flexible surgical instruments, which run parallel along the body of the laryngoscope and are fixed to the body of the laryngoscopes by three polylactide clamps flexibly attached to the body of the laryngoscope. This laryngoscope has the disadvantage that it does not have a high degree of mechanical stability due to the reversibly attached clamps and is expensive to manufacture. Furthermore, it is not possible with this laryngoscope to perform a surgical procedure by laser ablation, as the two working channels are not suitable for receiving and controlling a flexible laser fiber required for this. Furthermore, the structure of the laryngoscope restricts possibilities with regard to sterilization procedures and the laryngoscope cannot be easily fixed to a stable frame.

On this basis, the objective of the present invention was to provide a laryngoscope which overcomes at least one of the disadvantages known from the prior art, preferably all of the disadvantages known from the prior art. In particular, the laryngoscope should be easy and inexpensive to manufacture, be suitable for repeated use, be mechanically stable (also with respect to various sterilization methods), be easy and quick to handle (e.g. be easily and reversibly fixable to a stable frame and not require manual fitting with an imaging unit) and enable the use of a wide range of different, flexible surgical instruments, which can be easily moved to the site of the surgical procedure. The laryngoscope should enable the surgeon to use a bimanual surgical technique during the entire procedure.

The objective is achieved by the laryngoscope having the features of claim 1. The dependent claims give advantageous further developments.

According to the invention a laryngoscope is provided, comprising
a) a rigid, curved body, which has a proximal end and a distal end and extends from the proximal end to the distal end of the body in a longitudinal direction;
b) a handle which is connected to the proximal end of the body;
c) a curved extension, which is arranged on an upper section of the distal end of the body and is suitable for lifting the epiglottis of a patient;
d) a light source, which is arranged on a lower section of the distal end of the body and has or consists of one or more LEDs;
e) an image capturing unit, which is arranged on a lower section of the distal end of the body and comprises or consists of one or more image capturing chips;
f) two channels, which extend on opposite sides along the longitudinal direction of the body and which each have an opening at the proximal end and at the distal end of the body, wherein the two channels are suitable for receiving a flexible surgical instrument suitable for mechanical intervention at the site of surgery.

The laryngoscope according to the invention can be used for transoral surgical procedures in the larynx and pharynx.

The rigidity of the laryngoscope body has the advantage that a high degree of mechanical stability is ensured and the laryngoscope does not require time-consuming steps for stable positioning in the pharynx (e.g. no inflation of cushions for fixing, which would be necessary with a flexible body).

A rigid and straight laryngoscope would exert considerable force on oral and pharyngeal structures such as the teeth and tongue, in particular in difficult anatomical conditions or with insufficient cervical extension. This is mainly due to the conflict between the straight rigid form and curvature of the oropharyngeal corridor which often causes tissue trauma resulting in tooth fractures, hematomas or nerve injuries. The curved form of the body of the laryngoscope according to the invention has the advantage that significantly less force is exerted on the front teeth and there is no need for the cervical hyperextension of the patient during transoral procedures. This enables a gentler surgical option for patients, for whom transoral access is not possible and who would otherwise have to be operated on using an open more invasive approach involving a longer stay in hospital for example.

The handle enables easy handling of the laryngoscope. The handle is in particular shaped ergonomically, i.e. the handle is configured such that its form corresponds substantially to the shape of a hollow of a curved hand of a user.

The curved extension, which is suitable for lifting the epiglottis of a patient, has the advantage, that the tongue is retracted during the transoral application, allowing a better view of the site of the surgical procedure.

The arrangement of the light source and the image capturing unit at the distal end of the body of the laryngoscope has the advantage that the user can handle the laryngoscope more easily, quickly and without any complications, as there is no need to insert a light source and an image capturing unit before starting the surgery. The images from the image capturing unit can be visualized for the user by transferring the data from the image capturing unit to an external monitor. The image capturing unit can be a commercially available chip camera (in particular with a resolution of at least 640×480 pixels).

The two channels, which each extend on the opposite side along the longitudinal direction of the body of the laryngoscope (i.e. on the lateral sides of the body), have the advantage that each of the two channels can receive a flexible surgical instrument via which a mechanical intervention can be performed at the site of surgery. The two channels thus allow two flexible and manually controllable surgical instruments to be used for a mechanical intervention.

The laryngoscope can be characterized in that it comprises a further channel extending in the longitudinal direction of the body and having an opening at the distal end of the body and an opening in a direction of the proximal end of the body, wherein the further channel is suitable for receiving a flexible laser optical fiber (e.g. for coupling light from a CO₂ laser). The further channel can extend along an underside of the body of the laryngoscope, preferably along a central axis in the longitudinal direction of the body.

Preferably, the further channel has a control device at the distal end of the body, which is suitable for receiving a flexible laser optical fiber and controlling its movement. The advantage of this further channel is that not only can a flexible laser optical fiber be accommodated, but its movement can also be controlled. This enables the user to performed a targeted surgical intervention not only in a mechanical manner (i.e. requiring contact), but also in a contactless manner, i.e. to use the fine cutting properties of laser light during s surgery in a targeted and location-specific manner. The control device can be configured to mechanically and/or electrically control the movement of the laser optical fiber. In a configuration for mechanical control, the control device can have pull cords which can be operated by a user. In a configuration for electronic control, the control device can have piezo elements which are communicatively connected to a control device (e.g. a joystick). The control device particularly preferably has a diameter of 5-7 mm. A suitable control device is described for example in York, P.A. (“Microrobotic laser steering for minimally invasive surgery”, Science Robotics, 6:eabd5486, 2021).

The further channel preferably has a transparent disc at the distal end of the body (e.g. made of glass or a plastic that is transparent to laser light), which seals the channel fluid-tight at the distal end. The advantage of this is that liquids and other dirt cannot penetrate to the laser optical fiber and the control device during the surgery and therefore cannot damage them. Furthermore, the image capturing unit (e.g. at least one camera) and the light source (i.e. the at least one LED) can (respectively) be sealed by a light-transparent disc in order to prevent contamination of the electronic components.

The laryngoscope can be characterized in that it comprises or consists of a curved hook at a proximal end of the handle. The curved hook is particularly preferably curved in a direction of the body of the laryngoscope. The advantage of the curved hook is that the laryngoscope enables intraoperative fixing in a simple and stable manner. For example, the hook can be hooked securely onto a lateral telescopic extension (i.e. tube) of a commercially available fabric support in a simple and stable manner. This allows the entire laryngoscope to be fixed to a commercially available holder on the operating table.

The hook particularly preferably has a length in the range of 2 cm to 5 cm. Furthermore, the hook can have a width in the range of 1 cm to 3 cm. Apart from this the hook can have a curvature which can be described by a sector of a circle with a radius of 0.5 to 2 cm. A curvature in this range is advantageous for fixing to a lateral telescopic extension of a commercially available fabric support.

The laryngoscope can be characterized in that the rigid, curved body, the handle, and/or the curved extension (optionally also a curved hook of the laryngoscope) comprises or consists of a material selected from the group consisting of metal, plastic and combinations thereof, preferably stainless steel, titanium and/or aluminum. The advantage of a metal such as stainless steel, titanium and aluminum is that these materials are light, have high mechanical stability and can be reliably sterilized multiple times using standard sterilization methods without getting damaged.

In a preferred embodiment, the laryngoscope is suitable for sterilization with hydrogen peroxide, sterilization with ethylene oxide and/or a low-temperature chemical sterilization with peracetic acid without being mechanically damaged thereby. The advantage of this is that the laryngoscope is not a disposable object but can be reused multiple times, which provides an ecological and economic advantage.

The laryngoscope can be characterized in that the rigid, curved body, the handle and the curved extension (optionally together with a curved hook of the laryngoscope) are formed in one piece (monolithically). The advantage of being in one piece is that the laryngoscope is easy and quick to manufacture (e.g. by 3D printing) and that it is mechanically more stable than if the individual parts were simply fixed to one another (e.g. by an adhesive bond). In addition, the laryngoscope in this case has no joints or connecting points, where microorganisms can accumulate and impair the safe use of the laryngoscope in terms of its sterility.

The laryngoscope can be characterized in that the rigid curved body has a length in the range of 12 cm to 15 cm. The rigid, curved body can have a width in the range of 3 cm to 5 cm. Furthermore, the rigid curved body can have a curvature in an area which is connected to the handle which curvature can be described by a sector of a circle with a radius of 8 to 10 cm and which is formed away from the handle. Such a curvature has proved to be advantageous for handling the laryngoscope, as the handle arranged on the body is thus not inclined unnecessarily far towards the body. Furthermore, the rigid curved body can have a curvature in an area which is not connected to the handle, which curvature can be described by a sector of a circle with a radius of 6.5 to 9 cm and which is formed in a direction of the handle. Such a curvature is advantageous, as it allows the laryngoscope to be inserted more gently into the patient's throat.

The laryngoscope can be characterized in that the handle has a length in the range of 8 cm to 10 cm and/or a width in the range of 4 cm to 6 cm. These dimensions of the handle are advantageous, as they ensure a secure and firm grip of the laryngoscope.

The laryngoscope can be characterized in that the curved extension has a length in the range of 2 cm to 4.5 cm and/or a width in the range of 1.5 cm to 3 cm. Furthermore, the curved extension can have a curvature which can be described by a sector of a circle with a radius of 6 to 9 cm. These dimensions and/or this curvature have proved to be advantageous for lifting or pulling back a patient's tongue when placing the laryngoscope in the patent's throat, allowing a better view of the surgical site.

The laryngoscope can be characterized in that it has a further light source which is arranged in a lower section of the distal end of the body and has or consists of one or more LEDs, wherein the light source and the further light sources are preferably arranged at a distance of 0.5 to 1 cm from one another at the distal end of the body. The further light source has the advantage that the surgical area can be better illuminated. The distance between the two light sources has proven to be particularly advantageous, as the illumination can be better balanced by the other light source if one of the two light sources is partially covered in one area of the surgical site.

The laryngoscope can be characterized in that it comprises a further image capturing unit which is arranged in a lower section of the distal end of the body and comprises or consists of one or more image capturing chips, wherein the image capturing unit and the further image capturing unit are arranged at a distance of 1 to 2 cm from one another at the distal end of the body. The further image capturing unit can be used to improve the image capture of the surgical area, for example as three-dimensional imaging. The distance between the two image capturing units has proven to be particularly advantageous, as the image acquisition can be better compensated by the other image capturing unit if one of the two image capturing units is partly covered in one area of the surgical site. Apart from this, the distance between the two image capturing units makes it possible to acquire a 3D image of the surgical site, which can significantly increase the orientation in the surgical field and the precision of the surgery.

The laryngoscope can be characterized in that the light source and the image capturing unit are electrically conductively connected to an interface on a surface of the laryngoscope, preferably to an interface on a surface of the handle, wherein the interface is suitable for connection to a power source for the light source and image capturing unit. The interface allows a continuous power supply of the light source(s) and the image capturing unit(s) of the laryngoscope during a surgery.

The laryngoscope can be further characterized in that the light source and the image capturing unit are connected electrically conductively to an energy storage device (e.g. a primary battery, a secondary battery and/or a supercapacitor), which is arranged in an interior space of the laryngoscope, preferably in an interior space of the handle. The connection of the light source(s) and the image capturing unit(s) of the laryngoscope to an energy storage device in the interior space of the laryngoscope has the advantage that the light source(s) and the image capturing unit(s) are powered with electrical power by the laryngoscope itself, i.e. they can be operated autonomously.

The laryngoscope can be characterized in that it has an energy storage device, preferably in an interior space of the handle of the laryngoscope. The energy storage device in the interior space of the laryngoscope has the aforementioned advantages. The energy storage device can be suitable for charging via an interface which is arranged on a surface of the laryngoscope, preferably on a surface of the handle of the laryngoscope. Furthermore, the energy storage device can be suitable for wireless charging, preferably via induction. If suitable for wireless charging, there is no need for an interface on a surface of the laryngoscope to charge the energy storage device. This has the advantage that the sterilization of the laryngoscope is simpler and more reliable and prevents the accumulation of (pathogenic) microorganisms on the surface of the laryngoscope at an interface that would otherwise be necessary for charging.

The laryngoscope can be characterized in that the two channels, which extend on opposite sides in the longitudinal direction of the body, have, at least regionally, at least one, optionally several, slot(s) along their extension. The one or more slots provide at least one further opening of the two channels, in addition to the proximal and distal opening of the two channels. The advantage here is that the two channels can be cleaned and sterilized more reliably, as a liquid and/or a gas for cleaning or sterilization can penetrate better into the two channels and can act more reliably on each inner surface of the two channels. This enables a more economical, more sustainable and safer use of the laryngoscope. Furthermore, the at least one slot (optionally all slots) can enable a safe removal of material from the body of an operated patient (e.g. after a biopsy).

The at least one slot preferably has a length of at least 5 cm, wherein the at least one slot preferably extends over the entire length along the longitudinal direction of the body, in particular from the proximal end to the distal end of the body. The longer the slot, or the more short slots there are, the better it can be ensured that the cleaning and sterilization are thorough.

The at least one slot can have a width in the range of 0.2 cm to 0.6 cm perpendicular to the longitudinal direction of the body and/or substantially parallel to the longitudinal direction of the handle. A width in this range has proved to be advantageous, on the one hand, to ensure that cleaning and sterilization are thorough and, on the other hand, to ensure that a flexible surgical instrument inserted into the two channels does not leave the two channels laterally in an undesired manner, i.e. it is ensured that said instrument leaves the laryngoscope only at the distal opening of the respective two channels.

The laryngoscope can be characterized in that the two channels have a diameter in the range of 6 to 10 mm, preferably 6.5 to 9.5 mm. This diameter has proven to be advantageous for receiving common, flexible surgical instruments, as these often have a diameter of approximately 6 mm (e.g. DiLumen graspers, Lumedi, Westport, CT, USA).

The two channels are preferably suitable for receiving a flexible surgical instrument which is selected from the group consisting of grippers, scissors, electric needles, suction devices and combined suction-coagulation devices.

The laryngoscope can be characterized in that the two channels each have an opening at the distal end of the body, the minimum distance of which from an edge of the opening of one of the two channels to an edge of the opening of the other of the two channels is in the range of 2 cm to 3 cm. The advantage of a distance in this range is that it enlarges the surgical area that can be reached with the flexible surgical instruments inserted into the two channels. In addition, the large distance in this range enables improved triangulation of the flexible surgical instruments.

The two channels of the laryngoscope can run towards one another in the region of the distal end of the body (angulation), preferably at an angle in the range of 1 to 100 to an imaginary central axis of the body. This has the effect of improving the triangulation of the surgical instruments inserted into the two channels in the surgical area and therefore allows the two instruments to be placed more easily and precisely in the desired target location. For example, this angulation allows the grippers and scissors to converge more effectively on a central point of the surgical area.

With reference to the following Figures and the following example the subject-matter of the invention is explained in more detail, without wishing to limit it to the specific embodiments shown here.

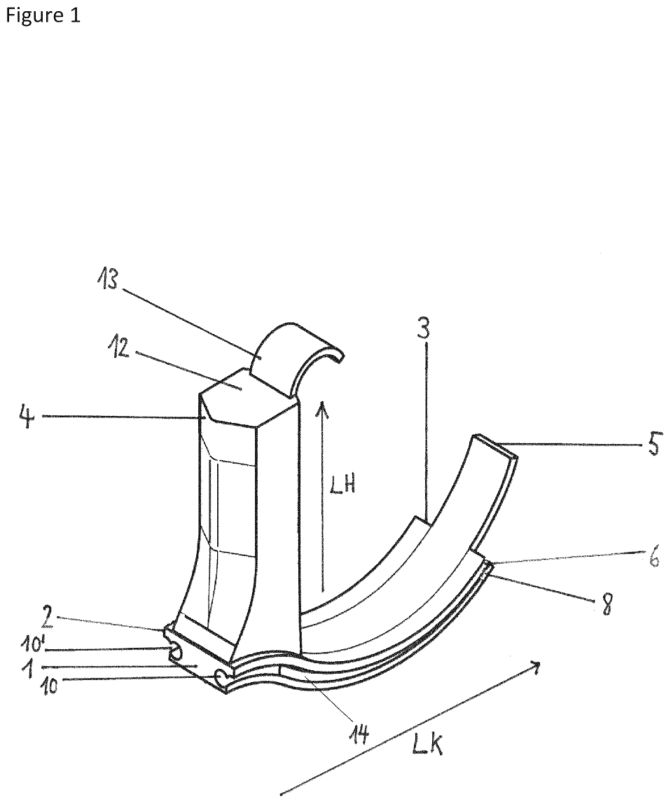

FIG. 1 shows schematically a laryngoscope according to the invention in an oblique side view with a view of the proximal end 2 of the rigid, curved body 1 of the laryngoscope. The rigid, curved body 1 of the laryngoscope extends from a proximal end 2 to its distal end 3. The laryngoscope has a handle 4 which is connected to the proximal end 2 of the body 1. Furthermore, the laryngoscope has a curved extension 5 (e.g. blade) which is arranged in an upper section 6 of the distal end 3 of the body 1 and is suitable for lifting the epiglottis of a patient. The upper section 6 at the distal end 3 of the laryngoscope is connected to a lower section 8 at the distal end 3 of the laryngoscope, wherein the upper section 6 and the lower section 8 are preferably formed in one piece (monolithically). Apart from this, the laryngoscope has two channels 10, 10′, which extend on respective opposite sides of the body 1 along the longitudinal direction LK of the body 1 and which each have an opening at the proximal end 2 and at the distal end 3 of the body 1, wherein the two channels 10, 10′ are suitable for receiving a flexible surgical instrument which is suitable for mechanical intervention at the site of surgery. In this embodiment the two channels 10, 10′ each have a slot 14 which allows a reliable cleaning and sterilization of the respective channels 10, 10′ and enables the safe removal of material from the body of an operated patient (e.g. after a biopsy). The slot 14 can have a width in the range of 0.2 to 0.6 cm, perpendicular to the longitudinal direction LK of the body 1 and/or substantially parallel to the longitudinal direction LH of the handle 1. In the embodiment shown here, a curved hook 13 is also arranged at the proximal end 12 of the handle 4, which enables an intraoperative fixing of the laryngoscope in a simple and stable manner.

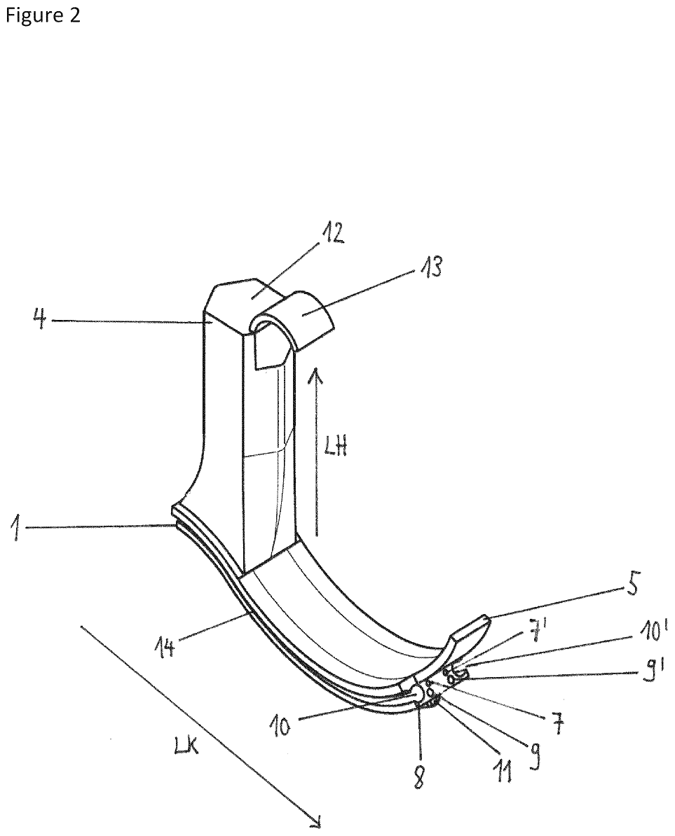

FIG. 2 shows schematically the laryngoscope according to the invention of FIG. 1 in an oblique side view looking towards the distal end 3 of the rigid, curved body 1 of the laryngoscope. In this view it can be seen that the laryngoscope on a lower section 8 of the distal end 3 of the body 1 has a light source 7 and here also a further light source 7′, wherein the light sources 7, 7′ each have or consist of one or more LEDs. Furthermore, it is clear in this view that the laryngoscope also has an image capturing unit 9 on the lower section 8 of the distal end 3 of the body and here too a further image capturing unit 9′, wherein the image capturing units 9, 9′ each have or consist of one or more image capturing chips. Furthermore, it can be seen in this view that the laryngoscope in this embodiment has a further channel 11 which extends in the longitudinal direction LK of the body 1 along an underside of the body 1 and has an opening at the distal end 3 of the body 1 and in a direction of the proximal end 2 of the body 1. The further channel 11 is suitable for accommodating a flexible laser optic fiber and controlling its movement, which enables the user to perform a targeted surgical intervention, not only in a mechanical way (i.e. requiring contact), but also in a contactless manner, i.e. to use the fine cutting properties of laser light in a targeted and site-specific manner during s surgery.

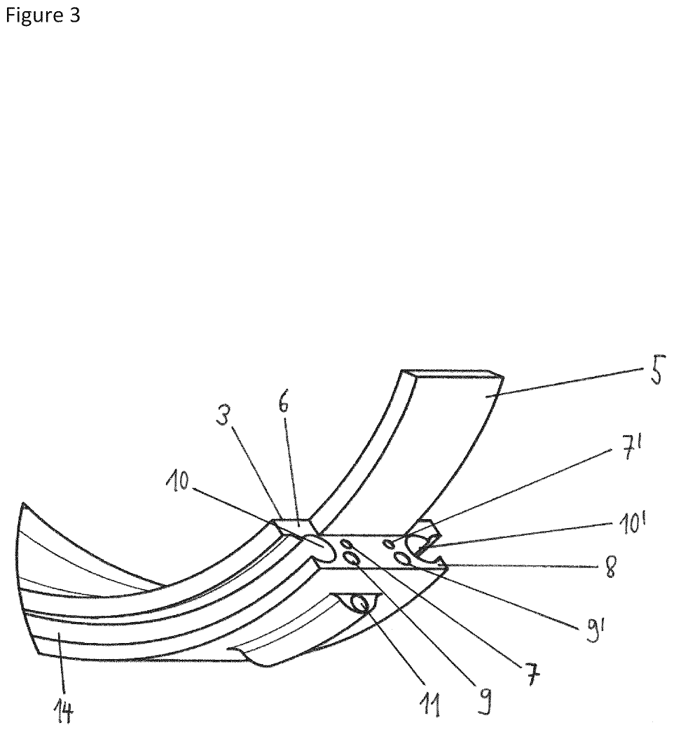

FIG. 3 shows schematically the distal end 3 of the rigid, curved body 1 of the laryngoscope from FIG. 1 in an enlarged view. In this view it is shown clearly that in this embodiment the light source 7 has a certain distance from the further light source 7′, which is preferably in the range of 0.5 to 1 cm. The distance between the two light sources 7, 7′ in this distance range has the advantage, that the illumination can be better balanced by the other light source 7, 7′ in the event of the partial covering of one of the two light sources 7, 7′ at a location of the operating area. Furthermore, it is clearly shown in this view that in this embodiment the image capturing unit 9 has a certain distance from the other image capturing unit 9′ which is preferably in the range of 1 to 2 cm. The distance between the two image capturing units 9, 9′ in this distance range has the advantage that image acquisition can be better balanced by the other image capturing unit 9, 9′ in the event of a partial covering of one of the two image capturing units 9, 9′ at a location of the operating area. Apart from this, the distance between the two image capturing units 9, 9′ makes it possible to acquire a 3D-image of the surgical area, which can significantly increase the orientation in the surgical field and the precision of the surgery. In this view in addition, the further channel 11 of the laryngoscope is also clearly visible, which is suitable for accommodating a flexible laser optical fiber and controlling its movement.

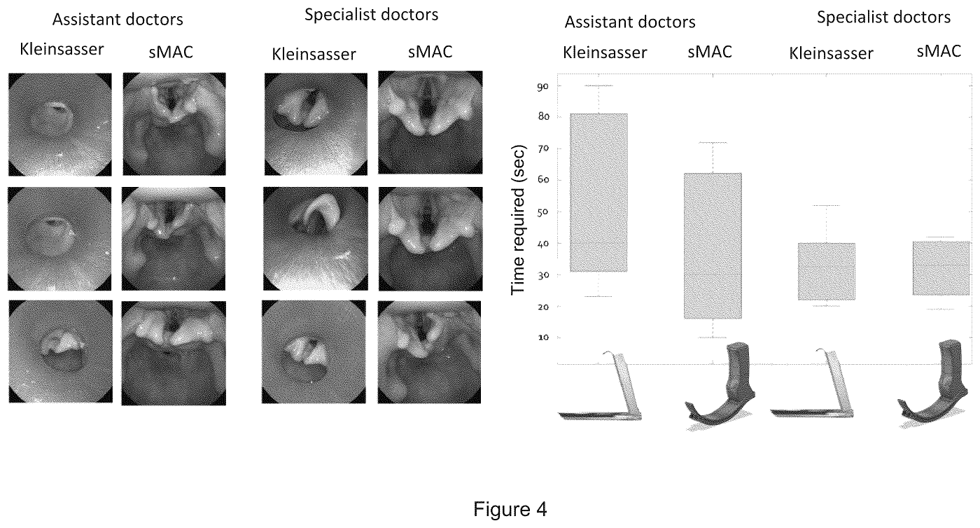

FIG. 4 shows the result of a visualization of the glottic plane with a laryngoscope (Kleinsasser tube) from the prior art (Kleinsasser) and a laryngoscope (sMAC) according to the invention by assistant doctors and specialist doctors respectively, wherein in the left-hand image the quality of the visualization of the glottic plane is shown and in the right-hand image the time required up to visualization of the glottic plane is shown.

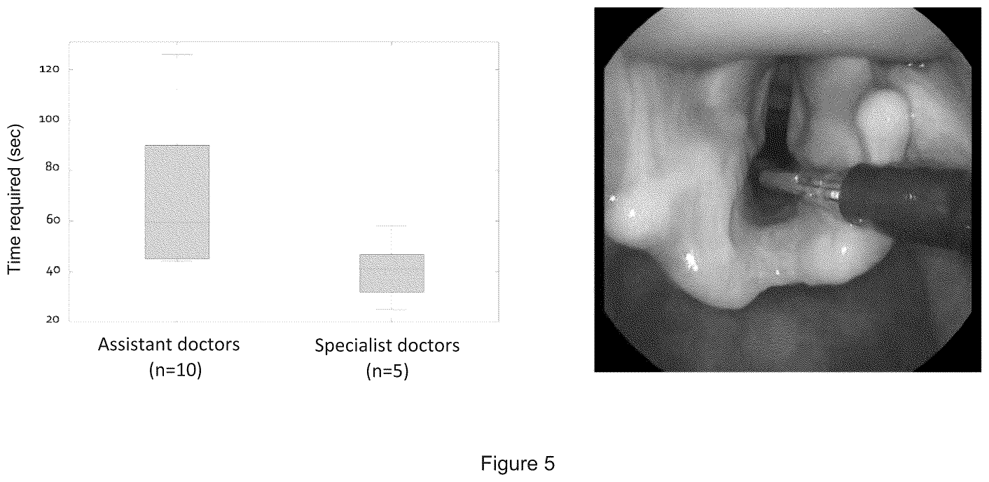

FIG. 5 shows the result of contacting laryngeal structures with the laryngoscope according to the invention by assistant doctors and specialist doctors respectively, wherein the time required up to contacting the laryngeal structures is indicated in each case.

### EXAMPLE—USE AND ADVANTAGES OF THE LARYNGOSCOPE ACCORDING TO THE INVENTION

Laryngoscopy with a laryngoscope, which has a rigid, straight (i.e. not curved) body (rigid laryngoscopy), has disadvantages (see below). Furthermore, a rigid laryngoscopy is often not adequate for patients with a high body mass index (BMI), degenerative changes in the cervical spine or postradiogenic trismus. For example, a rigid laryngoscopy is difficult to perform in the case of degenerative changes in the spine and trismus.

In fact, such a rigid laryngoscopy exerts considerable force on the patient's incisors (>50 N) and the laryngopharynx (>100 N), which can cause tongue pain, laryngeal edema, paralysis of the hypoglossal nerve, changes in taste (about 20%) and dental injuries (2-3%). Furthermore, it can contribute to deficient oncological treatment in patients.

The laryngoscope according to the invention which does not have a rigid, straight body, but has a rigid, curved body, can avoid these disadvantages.

The advantages of the laryngoscope according to the invention have been shown by a user study in which 10 assistant doctors and 5 specialist doctors at the ENT Clinic in Ulm had to perform the following tasks:
1. visualization of the glottic plane with rigid, straight laryngoscope (Kleinsasser tube) from the prior art (name: Kleinsasser) and a laryngoscope according to the invention (name: sMAC); and
2. Contacting laryngeal structures with the laryngoscope according to the invention.

Photo documentation was carried out and the time required to perform the respective tasks was measured.

The results shown in FIG. 4 demonstrate that the laryngoscope according to the invention (sMAC) enables an improved visualization of the glottic plane (see left image in FIG. 4) and at least assistant doctors with the laryngoscope according to the invention require significantly less time to visualize the glottic plane than with the laryngoscope from the prior art (Kleinsasser) (see right image in FIG. 4).

The result shown in FIG. 5 demonstrates that contacting laryngeal structures with the laryngoscope according to the invention requires only a very short time for both assistant doctors and specialist doctors, i.e. contacting laryngeal structures can be performed quickly.

### LIST OF REFERENCE SIGNS

1: rigid, curved body of the laryngoscope;
2: proximal end of the rigid, curved body of the laryngoscope;
3: distal end of the rigid, curved body of the laryngoscope;
4: handle;
5: curved extension (e.g. blade);
6: upper section of the distal end of the body of the laryngoscope;
7: light source;
7′: further light source;
8: lower section of the distal end of the body of the laryngoscope;
9: image capturing unit (one or more LEDs);
9′: further image capturing unit (one or more LEDs);
10, 10′: channel;
11: further channel;
12: proximal end of the handle;
13: curved hook;
14: slot in the channel;
LK: longitudinal direction of the rigid, curved body of the laryngoscope;
LH: longitudinal direction of the handle of the laryngoscope.

1-15. (canceled)

16. A laryngoscope comprising
a) a rigid, curved body, which has a proximal end and a distal end and extends from the proximal end to the distal end of the body in a longitudinal direction;
b) a handle which is connected to the proximal end of the body;
c) a curved extension, which is arranged on an upper section of the distal end of the body and is suitable for lifting the epiglottis of a patient;
d) a light source, which is arranged on a lower section of the distal end of the body and has or consists of one or more LEDs;
e) an image capturing unit, which is arranged on a lower section of the distal end of the body and includes or consists of one or more image capturing chips; and
f) two channels, which extend on opposite sides along the longitudinal direction of the body and which each have an opening at the proximal end and at the distal end of the body, wherein the two channels are suitable for receiving a flexible surgical instrument, which is suitable for a mechanical intervention at a site of surgery.

17. The laryngoscope according to claim 16, wherein the laryngoscope has a further channel, which extends in the longitudinal direction of the body and has an opening at the distal end of the body and in a direction of the proximal end of the body, wherein the further channel is suitable for receiving a flexible laser optical fiber.

18. The laryngoscope according to claim 16, wherein the laryngoscope comprises a curved hook at a proximal end of the handle.

19. The laryngoscope according to claim 18, wherein the curved hook is curved in a direction of the body of the laryngoscope.

20. The laryngoscope according to claim 19, wherein the curved hook has
i) a length in the range of 2 cm to 5 cm; and/or
ii) a width in the range of 1 cm to 3 cm; and/or
iii) a curvature which can be described by a sector of a circle with a radius of 0.5 to 2 cm.

21. The laryngoscope according to claim 16, wherein the rigid, curved body, the handle, and/or the curved extension, optionally also a curved hook of the laryngoscope, comprises a material selected from the group consisting of metal, plastic, and combinations thereof.

22. The laryngoscope according to claim 16, wherein the rigid, curved body, the handle and the curved extension, optionally together with a curved hook of the laryngoscope, are configured in one piece.

23. The laryngoscope according to claim 16, wherein the rigid curved body
i) has a length in the range of 12 cm to 15 cm; and/or
ii) has a width in the range of 3 cm to 5 cm; and/or
iii) in a region connected to the handle, has a curvature which can be described by a sector of a circle with radius of 8 to 10 cm and which is formed away from the handle; and/or
iv) in a region not connected to the handle, has a curvature which can be described by a sector of a circle with a radius of 6.5 to 9 cm.

24. The laryngoscope according to claim 16, wherein the handle
i) has a length in the range of 8 cm to 10 cm; and/or
ii) has a width in the range of 4 cm to 6 cm.

25. The laryngoscope according to claim 16, wherein the curved extension
i) has a length in the range of 2 cm to 4.5 cm; and/or
ii) has a width in the range of 1.5 cm to 3 cm; and/or
iii) has a curvature which can be described by a sector of a circle with a radius of 6 to 9 cm.

26. The laryngoscope according to claim 16, wherein the laryngoscope has a further light source, which is arranged on a lower section of the distal end of the body and which has one or more LEDs.

27. The laryngoscope according to claim 26, wherein the light source and the further light source are arranged at a distance of 0.5 to 1 cm from one another at the distal end of the body.

28. The laryngoscope according to claim 16, wherein the laryngoscope has a further image capturing unit, which is arranged on a lower section of the distal end of the body and comprises one or more image capturing chips.

29. Laryngoscope according to claim 28, wherein the image capturing unit and the further image capturing unit are arranged at a distance of 1 to 2 cm from one another at the distal end of the body.

30. The laryngoscope according to claim 16, wherein the light source and the image capturing unit
i) are connected electrically conductively to an interface arranged on a surface of the laryngoscope, wherein the interface is suitable for connecting to a power source for the light source and image capturing unit; and/or
ii) are connected electrically conductively to an energy storage device, which is arranged in an interior space of the laryngoscope.

31. The laryngoscope according to claim 16, wherein the laryngoscope has an energy storage device, wherein the energy storage device is optionally suitable
i) to be charged via an interface, which is arranged on a surface of the laryngoscope; and/or
ii) to be charged wirelessly.

32. The laryngoscope according to claim 16, wherein the two channels, which each extend on opposite sides along the longitudinal direction of the body, at least regionally have at least one, optionally a plurality of, slot(s) along their extension, which slot(s) provide(s) at least one further opening of the two channels in addition to the proximal and distal opening of the two channels.

33. The laryngoscope according to claim 32, wherein the at least one slot
i) has a length of at least 5 cm; and/or
ii) has a width in the range of 0.2 to 0.6 cm perpendicular to the longitudinal direction of the body and/or substantially parallel to the longitudinal direction of the handle.

34. The laryngoscope according to any claim 16, wherein the two channels
i) have a diameter in the range of 6 to 10 mm; and/or
ii) are suitable for receiving a flexible surgical instrument which is selected from the group consisting of electric needles, suction devices and combined suction-coagulation devices.

35. The laryngoscope according to claim 16, wherein the two channels
i) each have an opening at the distal end of the body, the minimal distance of which, from an edge of the opening of one of the two channels to an edge of the opening of the other of the two channels, is in the range of 2 cm to 3 cm; and/or
ii) run towards one another in the region of the distal end of the body.
